(19) United States
(12) Patent Application Publication
(10) Pub. No.: US 2025/0001041 A1
(43) Pub. Date: Jan. 2, 2025

(54) SULFONATION TREATMENT OF IMPLANTS

(71) Applicant: UNIVERSITY OF MISSISSIPPI MEDICAL CENTER, Jackson, MS (US)

(72) Inventors: Randall S. WILLIAMSON, Jackson, MS (US); Amol V. JANORKAR, Jackson, MS (US); Kadie NOBLES, Jackson, MS (US)

(21) Appl. No.: 18/756,172

(22) Filed: Jun. 27, 2024

Related U.S. Application Data
(60) Provisional application No. 63/523,624, filed on Jun. 27, 2023.

(51) Int. Cl. A61L 27/18; A61L 27/56

(52) U.S. Cl. CPC A61L 27/18; A61L 27/56; A61L 2400/18

(57) ABSTRACT

Disclosed is a method for treating a polyaryletherketone (PAEK) material to improve osseointegration properties of the PAEK material. The method includes: subjecting the PAEK material to a sulfonation process comprising contacting the PAEK material with sulfuric acid at a temperature and for a time sufficient to cause sulfonation of at least a portion of aryl groups of the PAEK material; following sulfonation and prior to exposing the PAEK material to water, subjecting the PAEK material to a physical cleaning process to remove at least a portion of excess sulfuric acid from the PAEK material; and subjecting the PAEK material to a hydrothermal process comprising contacting the PAEK material with water at a temperature and for a time sufficient to cause substantial de-sulfonation of the PAEK material. The resulting PAEK material includes porous surfaces that beneficially promote osseointegration when used in an implantable device.

### CROSS-REFERENCE TO RELATED APPLICATIONS

This application claims priority to and the benefit of U.S. Provisional Patent Application No. 63/523,624, filed Jun. 27, 2023 and titled “Sulfonation Treatment of Medical and Dental Implants,” the entirety of which is incorporated herein by reference.

### BACKGROUND

The need for orthopedic implants to treat musculoskeletal disorders has been on the rise and is expected to continue increasing as the age of the population increases. For example, the field has experienced a marked increase in spinal procedures in recent years. However, up to 10% of these implants experience failure due to causes such as poor osseointegration, infection, and failure/breakage. Similar issues persist with respect to other types of medical and dental implants intended for osseointegration. Osseointegration is the direct connection between living bone and the surface of an implant and is crucial for the long-term success of an implant.

Successful osseointegration into an implant interface is mainly influenced by material stiffness and porosity. To promote osseointegration, polymer materials are subjected to surface modification. Polyetheretherketone (PEEK) is a semi-crystalline non-resorbable polymer, which is radiolucent, biocompatible, and resistant to most chemicals. PEEK has gained popularity as an alternative replacement for titanium-based implants in orthopedic applications over recent decades. PEEK has an elastic modulus of 3-8 GPa, which is similar to the 7-30 GPa range of human cortical bone, and thus the problem of stress shielding often seen with titanium implants, which have an elastic modulus of 55-110 GPa, is diminished when using a PEEK-based material.

However, one disadvantage of utilizing PEEK is that the material is bioinert and integrates poorly without surface modifications to elicit desirable cellular responses. Accordingly, there is an ongoing need for improved surface treatments that can improve the properties of PEEK and similar polymers in the context of implantable devices.

### SUMMARY

Disclosed is a method for treating a polyaryletherketone (PAEK) material, such as PEEK, to improve osscointegration properties of the PAEK material. The method includes: subjecting the PAEK material to a sulfonation process comprising contacting the PAEK material with sulfuric acid at a temperature and for a time sufficient to cause sulfonation of at least a portion of aryl groups of the PAEK material; following sulfonation and prior to exposing the PAEK material to water, subjecting the PAEK material to a physical cleaning process to remove at least a portion of excess sulfuric acid from the PAEK material; and subjecting the PAEK material to a hydrothermal process comprising contacting the PAEK material with water at a temperature and for a time sufficient to cause substantial de-sulfonation of the PAEK material.

The resulting PAEK material includes porous surfaces that beneficially promote osscointegration when used in an implantable device. The implantable device can take any form where the intent is for the implant to undergo osscointegration following placement in a subject. Suitable implantable devices include, for example, spinal implants (e.g., spinal fusion cages/intervertebral spacers, spinal rods), orthopedic implants (e.g., joint replacement components, bone screws, plates, pins), dental implants (e.g., temporary abutments, healing caps, implant fixtures), and cranial/maxillofacial implants (e.g., skull plates, skull patches, maxillofacial implants).

The sulfonation process can include sonication, optionally with stirring, while the PAEK material is exposed to the sulfuric acid. The method can include a preliminary step of grinding a surface of the PAEK material and optionally washing the PAEK material prior to subjecting the PAEK material to the sulfonation process. The physical cleaning process can include using compressed air to remove at least a portion of the excess sulfuric acid from the PAEK material.

In one embodiment, the method comprises subjecting a PAEK material to a sulfonation process comprising contacting the PAEK material with sulfuric acid at a temperature of 55° C. to 75° C. and for a time of 1 to 3 minutes to cause sulfonation of at least a portion of aryl groups of the PAEK material; following sulfonation and prior to exposing the PAEK material to water, subjecting the PAEK material to a physical cleaning process to remove at least a portion of excess sulfuric acid from the PAEK material; and subjecting the PAEK material to a hydrothermal process comprising contacting the PAEK material with water at a temperature of 40° C. to 80° C. for a time of 1 to 5 hours to cause substantial de-sulfonation of the PAEK material.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is an illustration of a sulfonation reaction for forming sulfonated PEEK.

FIG. 2 is a reaction scheme showing sulfonation and de-sulfonation of PEEK.

FIG. 3 is a schematic illustrating de-sulfonation of residual sulfur groups during hydrothermal treatment of a previously sulfonated PEEK material.

FIGS. 4A and 4B are flowcharts illustrating example methods of treating a PAEK material to improve osseointegration properties of the PAEK material.

FIG. 5 shows PEEK specimens following an effective sulfonation process, showing resulting porous surfaces with effectively sized and distributed pores.

FIGS. 6A-6C are Fourier transform infrared spectroscopy (FTIR) graphs showing that hydrothermal treatment was effective in de-sulfonating tested specimens.

FIG. 7 shows surface roughness measurements of various specimens as measured using atomic force microscopy.

FIG. 8 shows contact angle measurements of various specimens.

FIG. 9 shows cell viability following osteoblast growth on various specimens according to an MTT assay.

FIG. 10A shows cell proliferation following osteoblast growth on various specimens, as measured by DNA content.

FIG. 10B shows cell differentiation following osteoblast growth on various specimens, as measured by ALP activity.

FIG. 11 shows images following Alizarin red staining of different specimen surfaces to observe cell maturation and mineralization.

FIG. 12 shows a PAEK specimen (non-PEEK material) after being subjected a sulfonation and hydrotreatment process, showing resulting porous surface with effectively sized and distributed pores.

### DETAILED DESCRIPTION

### I. Introduction

PEEK is a member of the larger category of polymers named polyaryletherketone (PAEK) polymers. PAEK materials are semi-crystalline thermoplastics with high-temperature stability and high mechanical strength, with a molecular backbone that contains alternately ketone (R—CO—R) and ether (R—O—R) groups. The linking group R between the functional groups is a 1,4-substituted aryl group. The aryl group can include, for example, phenyl, naphthyl, or biphenyl groups. All or some of the aryl groups can be substituted. Such substituents can include alkyl (e.g., methyl, ethyl) and/or halogen (e.g., chloro- or fluoro-) groups. While PEEK is the most common example of a PAEK polymer, the category includes other polymers, including polyetherketone (PEK), polyetherketoneketone (PEKK), polyetheretherketoneketone (PEEKK), and polyetherketoneetherketoneketone (PEKEKK). While certain embodiments and examples disclosed herein utilize PEEK as a specific example of a PAEK material, the skilled person will understand that the same principles and concepts disclosed herein are applicable to other PAEK materials.

Osseointegration is influenced by surface features such as roughness and wettability and is favorable to porous surfaces because they promote cellular attachment and allow cell infiltration. Osteoblasts range in size from 10 to 50 μm, but pore sizes used for orthopedic applications which have shown propitious osscointegration are in the size range of 100 to 400 μm. Surface roughness on the micro-scale, typically noted as 1-50 μm, can enhance cell adhesion and osseointegration by increasing early mechanical interlocking of the osteoblast cells, and roughness on the nano-scale of 1-1000 nm can act to signal cellular attachment and differentiation, since the roughness of natural bone is around 32 nm. A more hydrophilic surface, which allows for increased protein adsorption and favorable protein conformations, may improve cell attachment. However, studies of how wettability influences osseointegration have produced contradicting results in the literature, which shows surfaces ranging from superhydrophobic (water contact angle)>150° to superhydrophilic (water contact angle approaching) 0° to be optimal for attachment and growth of osteoblasts. The literature tends to point toward wettability being a secondary influence on osseointegration, whereas features such as porosity, roughness, and coatings are the primary influences.

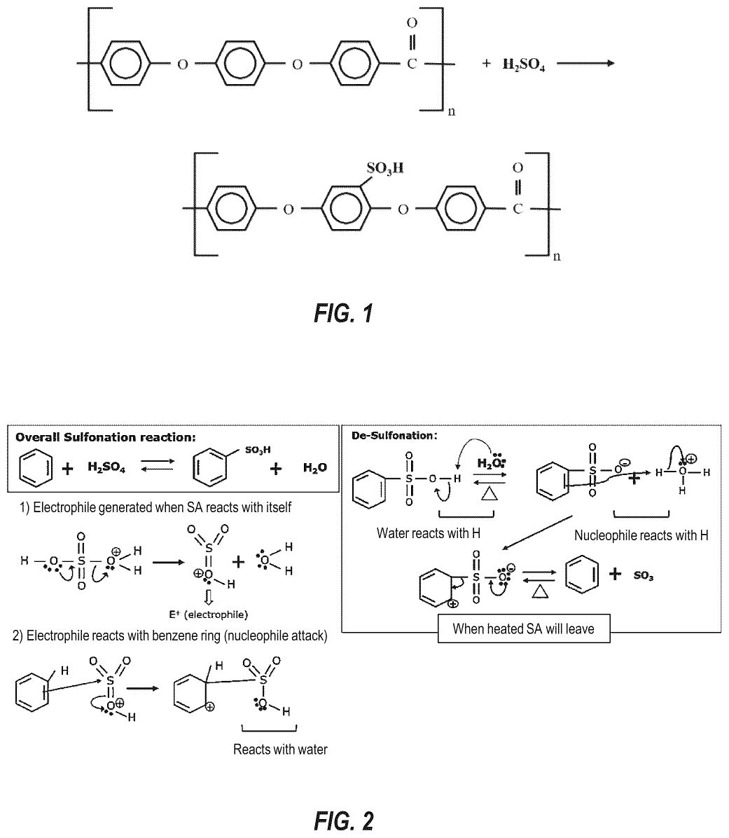

Sulfonation, which is the use of sulfuric acid to etch a surface, is one method to modify aromatic polymers. The porous network left behind on the surface of sulfonated PEEK can be beneficial in biomedical applications. PEEK sulfonation is an electrophilic reaction in which aryl groups (for PEEK, the hydroquinone unit benzene-1,4-diol, beside the ether bridge), is sulfonated, leaving behind a sulfonic acid, the —SO₃H group, as shown in FIG. 1.

This sulfuric acid etching creates a porous structure on the PEEK surface, which can be conducive to cellular attachment; however, the residual —SO₃H group left behind can be toxic in the physiological environment, thus needing to be removed through a de-sulfonation process. FIG. 2 is a reaction scheme showing typical sulfonation and de-sulfonation of PEEK.

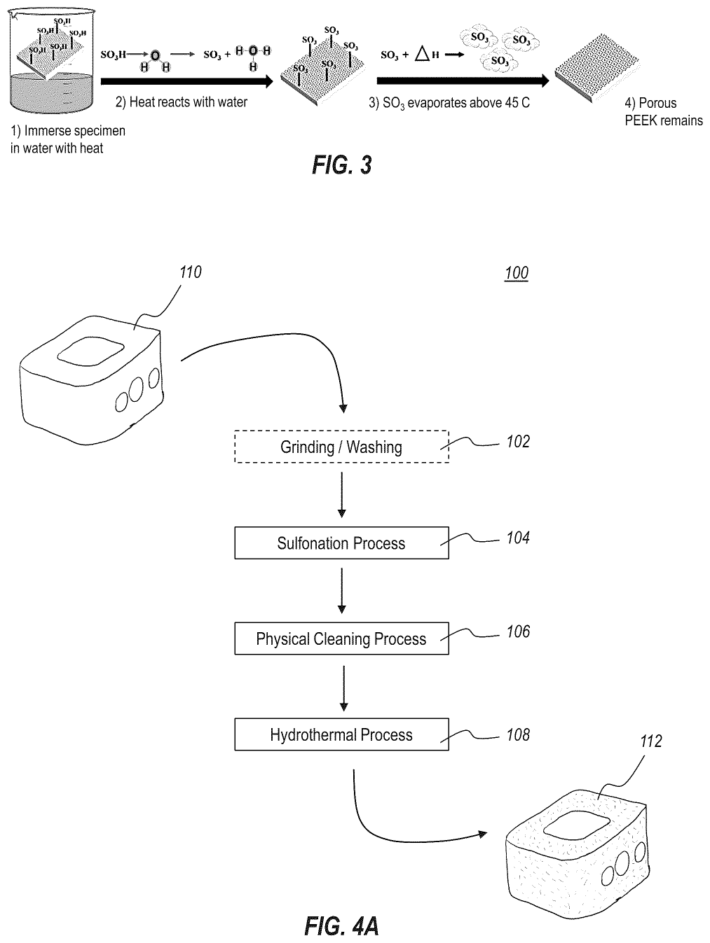

Hydrothermal treatment with water is one method that can be used to remove the residual —SO₃H groups. An overview of a hydrothermal treatment method is shown in FIG. 3. In the presence of water and heat, water molecules react with hydrogen (H) on the sulfur trioxide (SO₃) group, creating hydronium (H₃O), which reacts with the benzene ring, causing SO₃ to detach and leave as a gas when heated to its boiling point (about 45° C.).

### II. Enhanced Surface Treatment Process

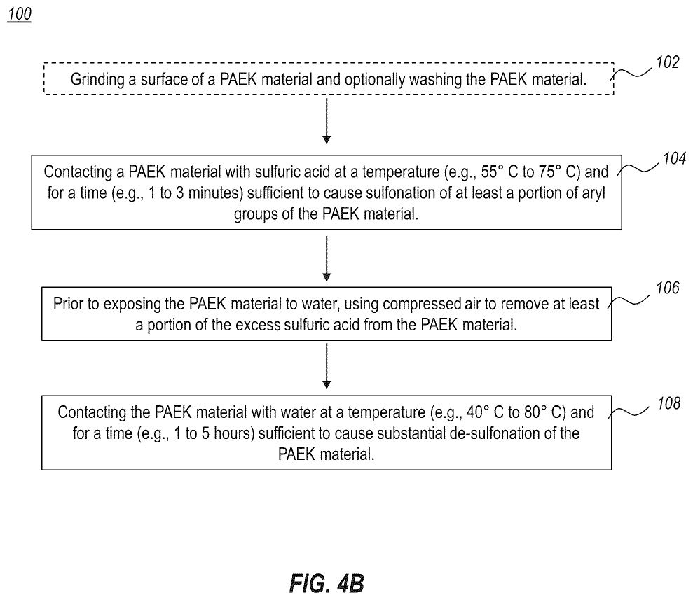

FIG. 4A is a flowchart illustrating an example method 100 for treating a PAEK material to obtain improved osseointegration properties. A starting PAEK material 110 can take the form of an implantable device. The implantable device can take any form where the intent is for the implant to undergo osseointegration following placement in a subject. Suitable implantable devices include, for example, spinal implants (e.g., spinal fusion cages/intervertebral spacers, spinal rods), orthopedic implants (e.g., joint replacement components, bone screws, plates, pins), dental implants (e.g., temporary abutments, healing caps, implant fixtures), and cranial/maxillofacial implants (e.g., skull plates, skull patches, maxillofacial implants).

FIG. 4A shows an example where the starting PAEK material is in the form of a spinal fusion cage, also referred to in the field as an interbody spacer, intervertebral body spacer, or simply as a spinal cage, or other similar terms. The disclosed methods can be carried out to provide effective PAEK modifications for any type of spinal fusion cage, including conventional spinal cages, expandable cages, and cages designed to accommodate bone graft material (allograft and/or autograft). Examples include those manufactured by Zavation Medical Products, LLC (Flowood, MS, USA), including the devices sold under the trade names Ti3Z CIF and Parallel T-PLIF PLIF.

The method 100 can optionally include a preliminary step of grinding a surface of the PAEK material and/or washing the PAEK material (step 102). The step of grinding the surface of the PAEK material can include using a grit paper rated at 220 grit to 440 grit, such as 240 grit to 420 grit, or 280 grit to 400 grit, or 320 grit to 360 grit, as defined by the Coated Abrasive Manufacturers Institute (CAMI) grit rating system, or a grit rating within a range with endpoints selected from any two of the foregoing values. As demonstrated in the below examples, preliminary grinding of PAEK material with grinding paper having a grit rating within the foregoing can beneficially contribute to effective pore size and pore coverage of treated PAEK material.

As shown, the method 100 includes a step of subjecting the PAEK material 110 to a sulfonation process that includes contacting the PAEK material 110 with sulfuric acid at a temperature and for a time sufficient to cause sulfonation of at least a portion of aryl groups of the PAEK material (step 104). Then, following sulfonation and prior to exposing the PAEK material 110 to water, the method 100 includes subjecting the PAEK material 110 to a physical cleaning process to remove at least a portion of excess sulfuric acid from the PAEK material (step 106). The method 100 then includes subjecting the PAEK material 110 to a hydrothermal process comprising contacting the PAEK material 110 with water at a temperature and for a time sufficient to cause substantial de-sulfonation of the PAEK material (step 108). The process 100 results in a surface treated PAEK material 112 with effective osseointegration properties.

The phrase “substantial de-sulfonation of the PAEK material” means that while a significant proportion of the SO₃H groups are removed, there may be some trivial, residual remnants that remain on the PAEK material, at least in some implementations. For example, “substantial de-sulfonation” may be taken to mean that no more than 1%, or no more than 0.1%, or no more than 0.01% of the aryl groups of the PAEK material remain sulfonated following hydrothermal de-sulfonation.

FIG. 4B is a flowchart showing a more detailed example of the method 100 for treating a PAEK material to obtain improved osseointegration properties. As discussed above, the method 100 can include an optional preliminary step of grinding a surface of the PAEK material and/or washing the PAEK material (step 102) prior to subjecting the PAEK material to the sulfonation process.

The sulfonation process can include contacting the PAEK material with sulfuric acid at a temperature and for a time sufficient to cause sulfonation of at least a portion of aryl groups of the PAEK material (step 104). For example, the sulfonation process can include contacting the PAEK material with sulfuric acid at a temperature of 55° C. to 75° C., or 60° C. to 70° C., or 65° C. (or a temperature within a range with endpoints selected from any two of the foregoing values) for a time of 1 to 3 minutes, such as no more than 2.5 minutes, or no more than 2 minutes. As demonstrated in the examples disclosed elsewhere herein, a sulfonation process carried out with such a temperature and duration can beneficially generate pores with size and coverage characteristics that are effective for promoting osseointegration.

The sulfonation process can be carried out with agitation, such as via sonication and/or stirring. Sonication was found to benefit the sulfonation process. Stirring or other mixing techniques may be used in addition or as an alternative to sonication.

The sulfuric acid used in the sulfonation process can have an acid concentration of greater than 80% (e.g., as an aqueous solution) and up to 100%. When acid concentrations of 80% or lower were used, the sulfonation process did not provide desired pores in the PAEK material. However, by using acid concentrations of greater than 80%, such as 100% sulfuric acid, the method 100 was effective in generating desired pores.

The physical cleaning process, carried out prior to exposing the PAEK material to water, can include using compressed air to remove at least a portion of the excess sulfuric acid from the PAEK material (step 106). The inventors discovered that the pores formed during the sulfonation process could be disrupted when the PAEK material was exposed to water following the sulfonation process due to an exothermic reaction between the sulfuric acid and water. Accordingly, while conventional hydrothermal treatment of sulfonated PAEK materials is beneficial in removing potentially cytotoxic —SO₃H groups, premature exposure to water can also disrupt the intended porous structure formed from the sulfonation process. The inventors discovered that a physical cleaning process can allow removal of sulfuric acid while preserving the porosity generated during the sulfonation step. The physical cleaning process can include, for example, subjecting the PAEK material to a jet of compressed air to substantially remove excess sulfuric acid remaining on the PAEK surface.

Negligible or trace amounts of sulfuric acid may remain even after a physical cleaning process. Accordingly, as used herein, “substantial removal of sulfuric acid” can mean removal of 60% or more, 70% or more, 80% or more, 90% or more, 95% or more, or 99% or more of excess sulfuric acid on the PAEK material.

The hydrothermal process can include contacting the PAEK material with water at a temperature and for a time sufficient to cause substantial de-sulfonation of the PAEK material (step 108). For example, the hydrothermal process can include contacting the PAEK material with water at a temperature of 45° C. to 80° C., or 50° C. to 70° C., or 55° C. to 65° C. (or a temperature within a range with endpoints selected from any two of the foregoing values) for a time of 1 to 5 hours, or 2 to 4 hours, or 3 hours, (or a time within a range with endpoints selected from any two of the foregoing values).

The method 100 can beneficially lead to a PAEK surface with a pore coverage (% area) of 5% or greater, such as 6% or greater, 8% or greater, 10% or greater, 11% or greater, 12% or greater, 14% or greater, 16% or greater, 18% or greater, and up to as high as 21%, such as a pore size within a range with endpoints selected from any two of the foregoing values. This level of pore coverage enables the treated PAEK material to effectively promote osseointegration relative to otherwise similar materials with lower levels of pore coverage. As used herein, the pore coverage (% area) can be calculated by taking a section of the treated PAEK surface and, from a plan view perspective above that section, determining the total surface area of the section and the surface area of the section covered by pores. The surface area of the pores divided by the total surface area of the section, expressed as a percentage, is the pore coverage (% area).

The method 100 can beneficially lead to a porous PAEK surface with pores having an average pore diameter (i.e., number average) of about 80 μm or greater, such as about 85 μm or greater, such as about 80 to about 120 μm. Pore sizes within this range are desired because they are within the range where effective osseointegration is encouraged upon implantation.

### III. Working Examples

The following examples illustrate a few example embodiments of the disclosed method and some resulting material properties. It will be understood that these examples do not limit the features of the disclosed method, nor do they fully define all results that could be expected from carrying out the disclosed method. For example, while the following testing identified a combination of parameters that exhibit effective results and that may form a presently preferred embodiment, other embodiments disclosed herein can also be utilized with one or more features that differ from the specific examples discussed below.

Testing was carried out to investigate the potential effects sulfonation processing may have on modifying the surface of PEEK for enhanced osseointegration. A hydrothermal treatment process was also utilized on the optimized sulfonated PEEK surface to remove the residual sulfocompound groups to render the surfaces advantageous to cellular attachment, proliferation, and differentiation. After successful optimization of the sulfonation process, an in vitro study with pre-osteoblast MC3T3-E1 cells was examined on the optimized PEEK surfaces though cell viability (MTT assay), cell proliferation (DNA assay), cell differentiation (ALP assay), and cell mineralization (Alizarin red assay).

### A. Materials & Methods

Materials: Medical-grade PEEK rods with approximately ½″ diameters were provided by Zavation Medical Products, LLC (Flowood, MS, USA). Concentrated sulfuric acid (95%-98%) was purchased from Sigma-Aldrich (Burlington, MA, USA). Silicon carbide grinding paper, grit 320, was purchased from Struers (Cleveland, OH, USA) for preparation of the PEEK surfaces before sulfonation.

Specimen Preparation: Disk-shaped specimens with a thickness of approximately 4 mm and a diameter of approximately 12.5 mm were cut from the PEEK rods using a Struers Accutom-50 sectioning saw (Cleveland, OH, USA). The disk specimens were then mounted in bakelite (Struers Citopress-20, Cleveland, OH, USA) and subsequently grinded for 15 s using a 320-grit silicon carbide grinding paper, washed, and grinded again for 15 s (Struers TegraPol, Cleveland, OH, USA). The specimens were removed from the mount and underwent a 5-stage wash cycle, consisting of the following: ultrasonic clean in Alconox® (Alconox Inc., White Plains, NY, USA) for 5 min, rinse with distilled water, ultrasonic clean in distilled water for 5 min, rinse with ethanol, and rinse with distilled water. The specimens were then allowed to air dry at room temperature and stored until further use.

Optimizing Sulfonated Surfaces: Investigated design parameters are shown in Table 1, and include presurface condition, soak condition, acid concentration, soak time, and soak temperature. Pre-surface condition refers to the roughness of the sulfonated surface obtained by using different grit sized silicon carbide grinding paper. The four soak conditions investigated include stirring(S), no stirring (NS), sonication only (SO), and sonication plus stirring (SS). Acid concentration refers to the concentration of the sulfuric acid used for the sulfonation process.

Initial testing indicated that a rougher surface was more conducive to initiating sulfonation on the PEEK surface; therefore, the high factor of 1200-grit grinding paper was eliminated. Stirring was also eliminated as a factor due to the creation of streaks on the sulfonated surface and addition of extraneous variables to control for in the process. The soak time high of 10 min resulted in zero pores on the surface and made the surface appear melted; therefore, soak time parameters were reduced significantly for subsequent testing. Additionally, acid concentrations lower than fully concentrated sulfuric acid did not react with the PEEK surfaces to create porosity.

TABLE 1
Investigated Sulfonation Design Parameters

| | Pre-Surface | | Soak | Acid | | | | | | |
|---|---|---|---|---|---|---|---|---|---|---|
| | (Grit) | | Condition | Conc. | Soak Time (min) | | | Soak Temp. (° C.) | | |
| Design | Low | High | S/NS/SO/SS | % | Low | Center | High | Low | Center | High |
| 1 | 220 | 1200 | S/NS | 80-100 | 1 | — | 10 | 22 | — | 60 |
| 2 | 220 | — | NS | 100 | 1 | 2.5 | 4 | 50 | 65 | 80 |
| 3 | 220 | — | NS | 100 | 2.5 | 3.75 | 5 | 55 | 65 | 80 |
| 4 | 320 | — | SO | 100 | 1 | 2.5 | 5 | 55 | 60 | 65 |
| 5 | 320 | — | SO/SS | 100 | 1 | 1.45 | 2.5 | 55 | 60 | 65 |
| 6 | 320 | — | SO/SS | 100 | 1 | — | — | 65 | — | — |

A brief surface roughness side study determined that a 320-grit grinding paper was optimal for matching surface roughness to as-manufactured solid PEEK and was therefore used for Design 4 to Design 6.

During design testing, a beneficial method for removing the sulfuric acid and cleaning the specimen after soaking was discovered. This method involved the use of compressed air to lightly blast the acid off the surface, which enabled retention of the pores that formed during the sulfonation process. It was discovered through experimentation that the pores formed during sulfonation could be disrupted when the specimen was cleaned after soaking due to the exothermic reaction that occurred when the sulfuric acid was mixed with water.

Each design had specimens sulfonated in random order to avoid any nuisance factors. Original non-treated PEEK has a yellowish-brown appearance in color, and once sulfonated, the surface appears white, but due to differences in lighting with the digital microscope and different surface features present on the specimens, all images were collected in the black and white color mode for pore measurements. ImageJ software Version 1.53e (National Institutes of Health, Bethesda, MD, USA) was used to extract the pore size data for comparison between the specimens.

Optimizing Hydrothermal Treatment: Hydrothermal treatment was conducted after optimized sulfonation factors were selected. The purpose of the hydrothermal treatment experiments was to determine steps in which most of the residual sulfocompound (sulfur) groups were no longer present on the sulfonated PEEK surface. The experiment was designed around a soak time low of 60 min and high of 90 min, and a soak temperature low of 45° C. and high of 80° C., as listed in Table 2. Two designs were conducted for the hydrothermal treatment testing. The hydrothermally treated specimens were sulfonated according to the parameters identified in Design 6 above. Water was continuously stirred during the hydrothermal treatment. The second design was chosen based on results from the first design. Fourier transform infrared spectroscopy (FTIR, Perkin-Elmer, Waltham, MA, USA) with a diamond/ZnSe crystal at a resolution of 4 cm⁻¹ and scanned from 650 to 4000 cm⁻¹ was utilized on smooth PEEK, sulfonated-only PEEK (sfPEEK), and sulfonated and heat-treated PEEK (sfPEEK-HT) to identify the functional groups of interest related to the successful removal of the residual sulfocompound groups from the sulfonated PEEK surface.

TABLE 2
Investigated Hydrothermal Treatment Design Parameters

| Design | Specimen | Soak Time (min) | Soak Temp. (° C.) |
|---|---|---|---|
| 1 | H1 | 90 | 45 |
| | H2 | 60 | 80 |
| | H3^{a} | 0 | 0 |
| | H4 | 75 | 63 |
| | H5 | 90 | 80 |
| | H6 | 60 | 45 |
| 2 | sfPEEK - 2 h HT | 120 (2 h) | 45 |
| | sfPEEK - 3 h HT | 180 (3 h) | |
| | sfPEEK - 4 h HT | 240 (4 h) | |
| | sfPEEK - 5 h HT | 300 (5 h) | |

^{a}H3 used as control specimen, i.e., sulfonated PEEK without HT treatment.
H3 and sfPEEK refer to the same condition herein.

Digital Imaging and Pore Size Measurements: All sulfonated surfaces were imaged and documented using a VHX digital microscope and its corresponding software (Keyence Corp., Osaka, Japan). Images were optimized using the contrasting and brightening features in Keyence before being processed using ImageJ software. ImageJ was used to measure the pore size and distribution data from the surfaces when applicable.

Atomic Force Microscopy: Atomic force microscopy (AFM, Bioscope Catalyst, Bruker, Santa Barbara, CA, USA) was performed on the optimized sulfonated and heat-treated specimens to determine the resulting surface roughness (Ra) values. The specimens were created in three batches (S1, S2, and S3) with n=2 (A and B) for each batch. Scans with 50 μm×50 μm area were acquired in ScanAssyst mode (0.100-0.25 Hz, and 512-256 samples/line) and further analyzed using Gwyddion software (version 2.41).

Contact Angle: Contact angle analysis was performed on the fully optimized surfaces from the AFM testing using 3 μL droplets of distilled water at ambient room temperature. Droplet images were captured using VHX digital microscopy (Keyence Corp., Osaka, Japan) and analyzed using Keyence software Version 1.2.0.2.

Cell Culture: MC3T3-E1 mouse pre-osteoblastic cells (American Type Culture Collection, Manassas, VA, USA) were maintained and expanded at 37° C. and 5% CO₂ in alpha-modified Eagle's minimum essential medium supplemented with L-glutamine, sodium pyruvate, 10% fetal bovine serum, and 1% penicillin-streptomycin, with the final pH adjusted to approximately 7.4. For experimentation, an osteogenic differentiation medium was formulated using alpha-modified Eagle's minimum essential medium supplemented with L-glutamine and sodium pyruvate, 10% fetal bovine serum, 1% penicillin-streptomycin, 0.284 mML-ascorbic acid, and 10 mM β-glycerophosphate. Approximately 50,000 cells/specimen were seeded and acclimatized for a day; thereafter, the specimens were supplemented with 1 mL of differentiation media every 48 h for a total of 21 days. The specimen types used for all in vitro experimentations were smooth PEEK, sfPEEK, sfPEEK-HT, with n=3 for each testing method. The sfPEEK-HT specimen was sulfonated according to sulfonation Design 6 detailed earlier. The smooth PEEK specimens were used as negative control in this study because it was anticipated that cells will not proliferate or readily mineralize on the smooth surface compared to the treated surface specimens.

Cell Viability: To assess cell viability at Day 7 and Day 21, a CyQuant™ MTT cell proliferation assay kit (ThermoFisher, Waltham, MA, USA) was used according to the manufacturer's protocol. Each specimen was incubated with 12 mM MTT stock solution and media for 4 h, followed by solution removal and addition of DMSO. Absorbance was read at 540 nm with an ELX-800 plate reader (Winooski, VT, USA).

Biochemical Analysis: Cells were trypsinized and collected off each specimen at the designated time points of Days 1, 7, 14, and 21 and stored at −80° C. until use. Cells were lysed via sonication for 1 min at 10% amplitude. DNA and alkaline phosphatase (ALP) assays were performed in triplicate.

Cell Proliferation: A CyQuant™ DNA cell proliferation assay (ThermoFisher, Waltham, MA, USA) was used according to the manufacturer's protocol on the lysed cells. Standard cell wells were conducted in duplicate. Fluorescence was measured at an excitation wavelength of 460 nm and emission wavelength of 520 nm on a Biotek FLx800 plate reader (Winooski, VT, USA).

Cell Differentiation: An alkaline phosphatase (ALP) assay was performed on the lysed cells to measure cellular differentiation. A QuantiChrom ALP assay kit (BioAssay Systems, Hayward, CA, USA) was used according to the manufacturer's protocol at an absorbance of 405 nm on an ELX-800 plate reader.

Cell Mineralization: To verify mineralization of the pre-osteoblasts on each specimen, calcium deposition was visualized using Alizarin red staining via osteogenesis quantitation kit (EMD Millipore, Billerica, MA, USA) according to the manufacturer's protocol. A control specimen containing no cells for each specimen type was analyzed as well. A VHX digital microscope was used to image the mineralized staining.

Statistical Analysis: Welch ANOVA (α=0.05) was used to determine any differences among the specimen groups in terms of pore size measurement, AFM, contact angle, mechanical testing, and in vitro experiments. For statistical comparison among the specimen groups, a Dunnett post hoc test was performed. Normality was checked using a Shapiro-Wilk test before calculating the ANOVA. For the specimen groups with extremely large counts for pore size measurements, any slight variations in normality were ignored due to ANOVA being robust against deviations in normality, resulting in a small effect on Type I error rate. All statistical analyses were performed using GraphPad Prism Software (version 8.3.0).

### B. Results

Optimized Sulfonation: Measurable surface porosity was not achieved in the first few designs, leading to each experiment being scored qualitatively by visually examining the surface and determining which surface experienced changes, such as the porosity present, similar porosity distribution across the surface, and an even sulfonated surface texture. The parameters from the most visually desirable surfaces on the specimen(s) from each design were chosen, and subsequent designs were built around those factors until an effective surface was attained. As discussed above, it was discovered that blasting the sulfonated surface with compressed air allowed the removal of sulfuric acid while preserving the porosity formed during sulfonation.

Design 6 was performed with the preferred time and temperature identified from Design 5 but with the soak condition of sonication only and sonication plus stirring under investigation. The results from this design showed similar pore surface coverage, pore count, and pore diameter, as shown in Table 3. Since similar results were obtained across the specimen types, the inventors moved forward using the sonication-only method, since this was more controllable.

TABLE 3
Porosity data from specimens in Design 6

| | | | Pore Diameter |
|---|---|---|---|
| | Pore Coverage | | (Feret diameter) |
| Specimen | (% area) | Pore Count | (Avg: μm) |
| D6-1-SS | 13 | 2124 | 103 ± 128 |
| D6-2-SS | 17 | 4179 | 88 ± 97 |
| D6-3-SS | 11 | 2531 | 94 ± 92 |
| D6-1-SO | 11 | 1574 | 120 ± 109 |
| D6-2-SO | 18 | 2558 | 115 ± 151 |
| D6-3-SO | 21 | 2671 | 118 ± 160 |

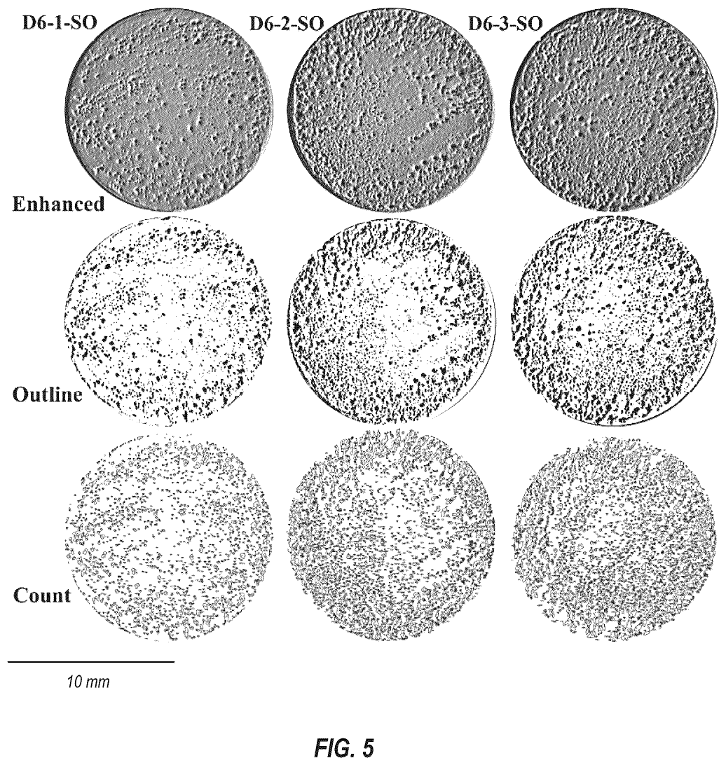

FIG. 5 shows the specimens from Design 6. The images were enhanced in black and white for easier identification of the pores using ImageJ software. The preferred sulfonation parameters obtained from Design 6 were a pre-surface condition using a 320-grit grinding paper, sonication-only soak method, soak time of 1 min, and soak temperature of 65° C.

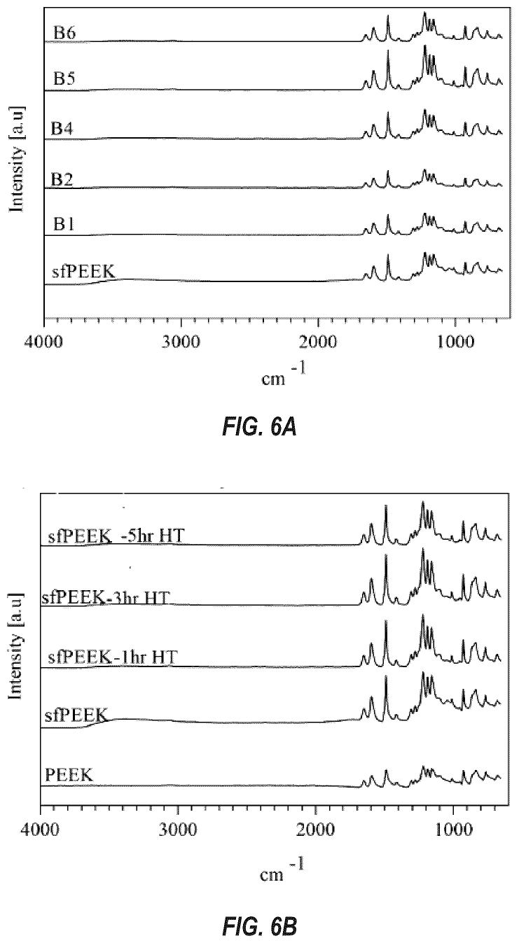

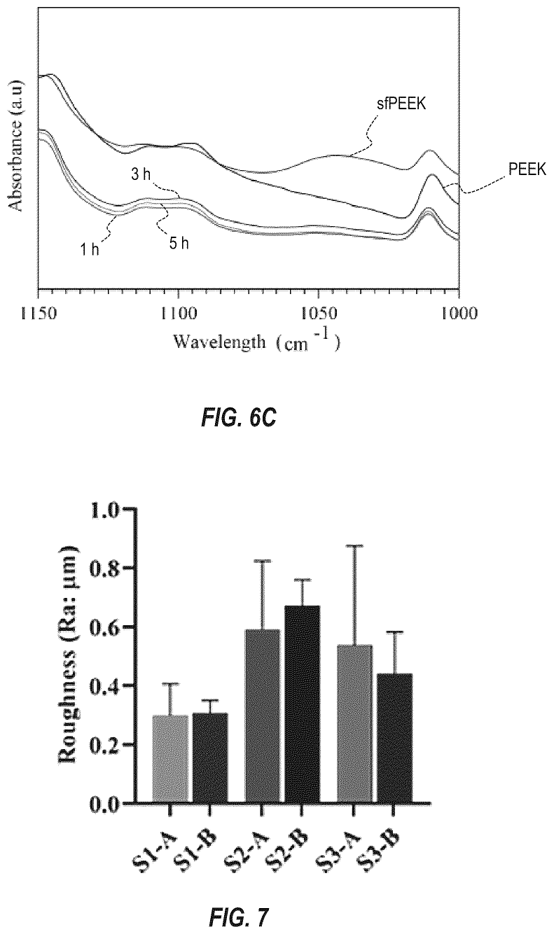

Hydrothermal Treatment: Digital imaging of the specimens before and after HT showed no evidence of changing surface features, indicating that the HT only removes residual sulfocompound groups and does not change surface morphology. FTIR was performed on each sample to identify the functional groups of interest. The spectra for samples from the hydrothermal treatment Design 1 are shown in FIG. 6A. In the graphs, sfPEEK refers to the positive control specimen, which is sulfonated PEEK without hydrothermal treatment, and the specimen labeled PEEK is the negative control, comprising untreated PEEK. In FIG. 6A, there is a large, broad peak around 3400 cm⁻¹ on the sfPEEK specimen, indicating the presence of the residual —SO₃H group, and another peak at approximately 1070-1100 cm⁻¹, which represents O═S═O stretching. The other specimen types in FIG. 6A exhibit decreasing intensities of the peaks at 3400 and 1070 cm⁻¹. These data suggest that the removal of residual sulfur groups appears more dependent upon time than temperature. Therefore, Design 2 utilized a set temperature of 45° C. and adjusted the time to investigate the time points shown in Table 2. The FTIR data from the hydrothermal treatment Design 2 are shown in FIGS. 6B-6C. The 2 h and 4 h time points were removed to better visualize the results and not overcrowd the graphs. FIG. 6C shows the results on the same baseline showing wavelengths of interest in the range of 1000-1150 cm⁻¹, which is the location of the S═O vibration peak. The results reveal no peak in the 3400 cm⁻¹ range and no peak in the 1070 cm⁻¹ range on the untreated PEEK specimen and significant peaks on the sfPEEK specimen. The data show lower intensities of the peaks of interest for HT treated specimens, but similar values are noted at 1, 3, and 5 h, suggesting that a hydrothermal treatment performed at 45° C. in the range of 1-5 h is sufficient for effectively reducing residual sulfur compound groups.

Atomic Force Microscopy: The specimens were created in three batches (S1, S2, and S3), with n=2 (A and B) for each batch, as discussed in the methods section above. The surface roughness of the sulfonated PEEK specimens is very tortuous, with varying degrees of topography, as can be seen in the wide range of roughness values shown in FIG. 7. However, across each specimen, there was no significant difference in the mean roughness values, which were 0.298±0.108 μm, 0.308±0.042 μm, 0.592±0.231 μm, 0.671±0.089 μm, 0.538±0.337 μm, and 0.440±0.143 μm for specimens S1-A-S3-B. There were 15 different statistical comparisons for these specimens, and each had a p<0.05, indicating no significant difference.

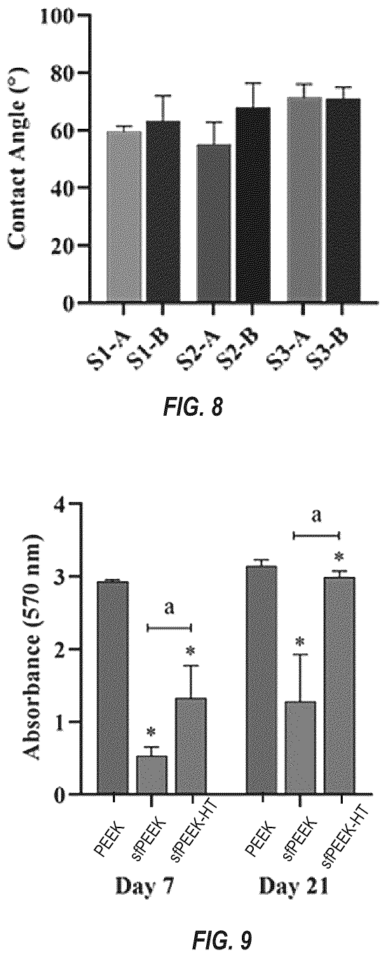

Contact Angle Analysis: FIG. 8 shows that all specimens recorded hydrophilic contact angles below 90°, and there was no significant difference among the specimens. The lowest recorded mean was 59.62°, and the highest was 71.47°.

Cell Viability: An MTT assay was performed on the cultured specimens after 7 and 21 days. FIG. 9 shows significantly higher cell viability for PEEK specimens over sfPEEK (p-value≤0.0001) and sfPEEK-HT (p-value≤0.0001) after Day 7, and for PEEK specimens over sfPEEK (p-value≤0.0001) and sfPEEK-HT (p-value=0.0024) on Day 21. The sfPEEK-HT specimen had higher viability compared to sfPEEK on Day 7 (p-value=0.0017) and on Day 21 (p-value=0.0001). In FIG. 9, significance at p<0.05 is shown with * as compared to PEEK and a as compared to sfPEEK.

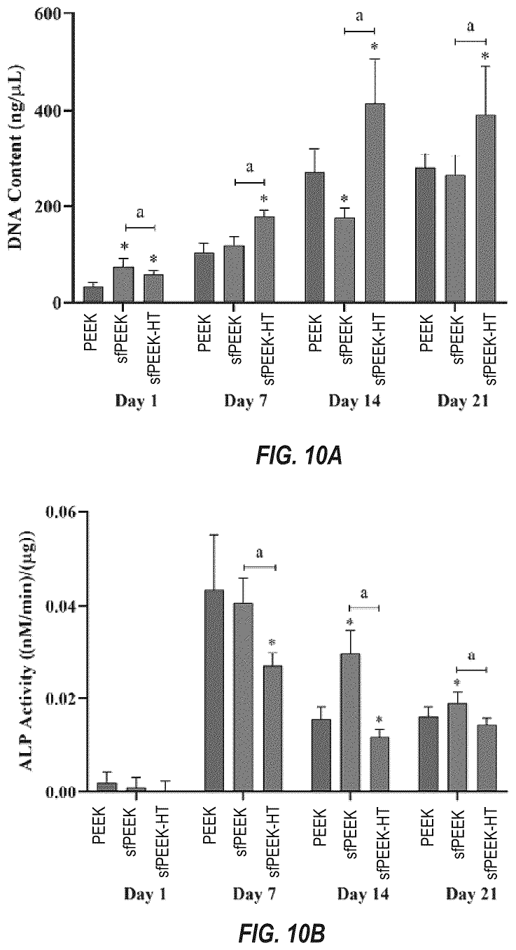

Cell Proliferation and Differentiation: Cell proliferation and differentiation on Days 1, 7, 14, and 21 were measured using a DNA and ALP assay kit, respectively. FIG. 10A shows increasing cell proliferation for all specimens across each time point. Significantly more DNA content was measured for sfPEEK-HT on Day 7 (p-value≤0.0001), Day 14 (p-value=0.0029), and Day 21 (p-value=0.0301) compared to PEEK, and significantly more DNA was measured for sfPEEK-HT on Day 7 (p-value≤0.0001), Day 14 (p-value≤0.0001), and Day 21 (p-value=0.0136) compared to sfPEEK. Both sfPEEK and sfPEEK-HT had significantly higher cell proliferation than PEEK on Day 1 (p-value≤0.0001). However, PEEK had significantly higher values on Day 14 compared to sfPEEK (p-value=0.0011). FIG. 10B shows low ALP activity on Day 1 for all specimens, but then, a large increase in ALP activity for all specimens occurs by Day 7. PEEK (p-value=0.0072) and sfPEEK (p-value≤0.0001) have significantly higher ALP activity than sfPEEK-HT on Day 7. Additionally, PEEK (p-value=0.0063) and sfPEEK (p-value≤0.0001) have significantly higher ALP activity on Day 14 compared to sfPEEKHT. ALP activity by Day 21 is still significantly higher for sfPEEK compared to the other two specimen types: PEEK (p-value=0.0337) and sPEEK-HT (p-value=0.0005). In FIGS. 10A and 10B, significance at p<0.05 is shown with * as compared to PEEK and a as compared to sfPEEK.

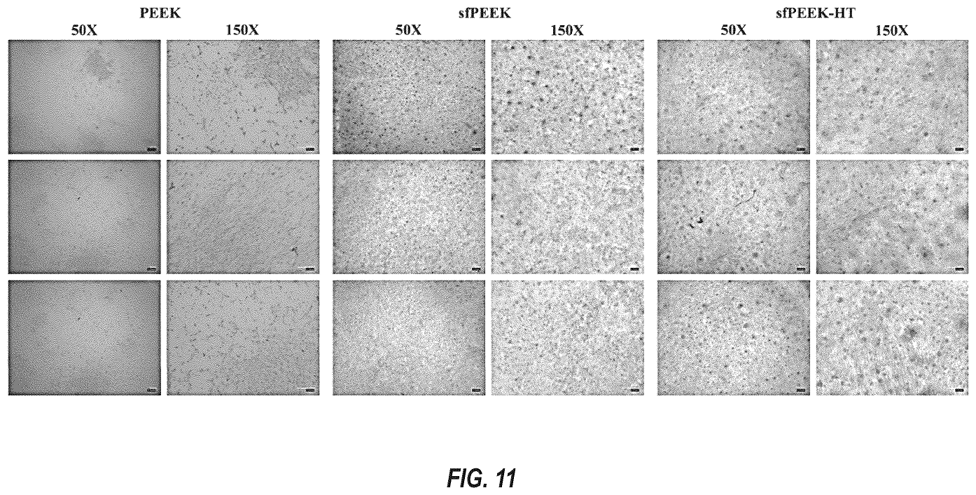

Cell Mineralization: The Alizarin red staining technique and subsequent digital imaging were performed to determine whether the osteoblast cells matured and mineralized on the different specimen surfaces. Controls were used to verify that the surfaces did not uptake any of the stain and interfere with cell identification. Alizarin red stains calcium deposits from mature osteoblasts. As shown in FIG. 11 (n=3 samples of each specimen type), there was limited staining on the PEEK specimens and copious amounts for the sfPEEK and sfPEEK-HT specimen types. This indicates more overall cell differentiation and mineralization for the sfPEEK and sfPEEK-HT specimen types compared to PEEK.

### C. Other (Non-PEEK) PAEK Materials

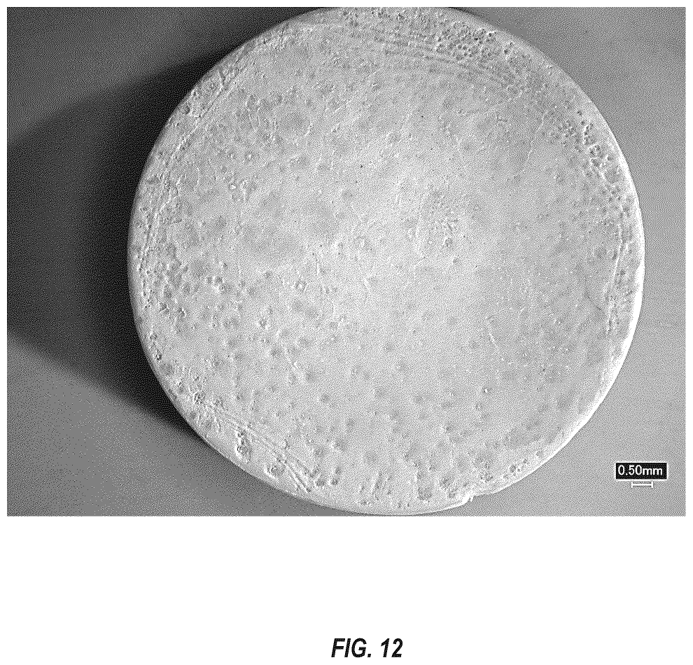

A specimen formed from a PAEK material, as sold under the trade name AVASPIRE AV-621 NT (Solvay Specialty Polymers USA, LLC), was subjected to the optimized sulfonation and hydrothermal treatment process used in the foregoing examples. That is, sulfonation Design 6 and hydrothermal treatment design 2, with at least one hour soak time, were applied to the PAEK specimen. The PAEK material is not specified by the manufacturer but is known to be a PEEK alternative within the larger class of PAEK materials. The resulting specimen is shown in FIG. 12. As shown, the treated PAEK material exhibits a porous surface with pores that are well sized and well distributed across the specimen surface. Although further pore characterizations were not carried out, these preliminary results indicated that the disclosed method is effective for treating other PAEK materials in addition to PEEK and is therefore expected to show efficacy with any suitable PAEK material.

### D. Discussion

The most effective sulfonation results, in this set of tests, were achieved using a soak time of 1 min and a soak temperature of 65° C. We also determined that the soak condition can effectively utilize either sonication or sonication with stirring. However, a more efficient and controllable sulfonation process can use sonication only (without stirring) with effective results. Using air to blast away sulfuric acid before rinsing is a beneficial method to clean the specimens after sulfuric acid soak. These factors resulted in pores on the sfPEEK surface ranging from 85 to 108 μm in average size, which is within the 100-400 μm pore size range considered conducive to osteoblast attachment and growth.

The porosity distribution across the surfaces of sulfonated PEEK was visually well dispersed, as shown in FIG. 5. Specimen types varied statistically when observing the pore sizes as a whole and in the 0-150 μm range. However, when the pore size range was narrowed to a range between 151 and 400 μm, there were no significant differences among the sulfonated PEEK specimens. Similar sizes between 151 and 400 μm are beneficial because a pore size of at least 100 μm is considered a major influencing factor in enhancing osteogenesis. Furthermore, the natural structure of cortical bone ranges in pore size from 10 to 500 μm.

The hydrothermal treatment experiments suggested that soaking sfPEEK in distilled water at 45° C. creates surfaces conducive to cellular growth. The hydrothermal treatment experiments conducted for this study ranged in temperature from 45° C. to 80° C., showing no significant difference in sulfur concentration based on temperature. Rather, notable differences in residual sulfur content were based on soak time. This indicates that a minimal temperature of 45° C. is adequate for removing sulfur content from the sulfonated PEEK, which will promote cell viability.

FTIR results show higher intensities of the target peaks for specimens which were not hydrothermally treated compared to those which were hydrothermally treated, which indicates the success of the treatment in removing residual sulfur groups. This is further supported by the contact angle analysis displaying a significantly higher contact angle for sfPEEK-HT, which would be more hydrophobic due to absence of OH— group from the —SO3H, compared to sfPEEK.

AFM results gave Ra values ranging from 0.298 to 0.671 μm. This changing topography indicates the presence of micro- and submicro-structures. This roughening of the PEEK surface following sulfonation is conducive to osteoblast adhesion, proliferation, and mechanical interlocking.

The MTT testing revealed the sfPEEK-HT specimen to have significantly higher viability than sfPEEK over the 21 days. Overall, sfPEEK-HT has the most significant and highest increase in DNA content over the course of the 21 days, indicating the highest cell proliferation and presence for that surface. The lower values of DNA content seen for the sfPEEK specimens could indicate that the cells on that surface have switched from proliferation to differentiation because the ALP activity for sfPEEK on Day 7 is significantly higher than sfPEEK-HT and significantly higher than PEEK and sfPEEK-HT on Days 14 and 21.

The mineralization seen in the Alizarin red staining further confirms the switch from proliferation to differentiation, which occurred on the specimen surfaces, as indicated by the ALP activity. Even though the PEEK specimens have a large increase in ALP activity on Day 7, which would indicate differentiation, the DNA content is low compared to the other specimen types, meaning that the overall cell count on the surface could be lower, resulting in the lower overall mature osteoblast formation seen. In contrast, the sfPEEK and sfPEEK-HT surfaces have an abundant amount of staining. Visually, it is difficult to determine whether sfPEEK or sfPEEK-HT have more mineralization; however, the DNA and ALP data suggest that cells proliferate more on sfPEEK-HT and eventually differentiate and mineralize, while the cells on sfPEEK proliferate less and differentiate earlier.

### IV. Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

In addition, unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, surface treatment steps and/or other surface-applied chemicals that are not specifically disclosed herein may optionally be completely omitted or essentially omitted from the disclosed embodiments.

An embodiment that “essentially omits” or is “essentially free of” a component may include trace amounts and/or non-functional amounts of the component. For example, an “essentially omitted” component may be included in an amount no more than 1%, no more than 0.1%, or no more than 0.01% by total weight of the composition. This is likewise applicable to other negative modifier phrases such as, but not limited to, “essentially omits,” “essentially without,” similar phrases using “substantially” or other synonyms of “essentially,” and the like.

A composition that “completely omits” or is “completely free of” a component does not include a detectable amount of the component (i.e., does not include an amount above any inherent background signal associated with the testing instrument) when analyzed using standard analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

1. A method of treating a polyaryletherketone (PAEK) material to improve osseointegration properties of the PAEK material, the method comprising:
subjecting a PAEK material to a sulfonation process comprising contacting the PAEK material with sulfuric acid at a temperature and for a time sufficient to cause sulfonation of at least a portion of aryl groups of the PAEK material;
following sulfonation and prior to exposing the PAEK material to water, subjecting the PAEK material to a physical cleaning process to remove at least a portion of excess sulfuric acid from the PAEK material; and
subjecting the PAEK material to a hydrothermal process comprising contacting the PAEK material with water at a temperature and for a time sufficient to cause substantial de-sulfonation of the PAEK material.

2. The method of claim 1, wherein the sulfonation process comprises contacting the PAEK material with sulfuric acid at a temperature of 55° C. to 75° C. and for a time of 1 to 3 minutes.

3. The method of claim 1, wherein the sulfonation process further comprises sonication, optionally with stirring, while the PAEK material is exposed to the sulfuric acid.

4. The method of claim 3, wherein the sulfonation process does not include stirring of the sulfuric acid.

5. The method of claim 1, wherein the hydrothermal process comprises contacting the PAEK material with water at a temperature of 45° C. to 80° C. for a time of 1 to 5 hours.

6. The method of claim 1, wherein the physical cleaning process comprises using compressed air to remove at least a portion of the excess sulfuric acid from the PAEK material.

7. The method of claim 1, wherein the PAEK material comprises one or more of polyetheretherketone (PEEK), polyetherketone (PEK), polyetherketoneketone (PEKK), polyetheretherketoneketone (PEEKK), or polyetherketoneetherketoneketone (PEKEKK).

8. The method of claim 7, wherein the PAEK material consists essentially of PEEK.

9. The method of claim 1, further comprising grinding a surface of the PAEK material and optionally washing the PAEK material prior to subjecting the PAEK material to the sulfonation process.

10. The method of claim 9, wherein grinding the surface of the PAEK material comprises using a grit paper rated at 220 grit to 440 grit.

11. The method of claim 1, wherein the sulfuric acid used in the sulfonation process has an acid concentration greater than 80% and up to 100%.

12. The method of claim 1, wherein a treated surface of the PAEK material exhibits a pore coverage (% area) of 5% or greater as a result of the method.

13. The method of claim 1, wherein a treated surface of the PAEK material exhibits an average pore diameter of 80 μm or greater as a result of the method.

14. An implantable device comprising a PAEK material manufactured using the method of claim 1.

15. A method of treating a polyaryletherketone (PAEK) material to improve osseointegration properties of the PAEK material, the method comprising:
subjecting a PAEK material to a sulfonation process comprising contacting the PAEK material with sulfuric acid at a temperature of 55° C. to 75° C. and for a time of 1 to 3 minutes to cause sulfonation of at least a portion of aryl groups of the PAEK material, wherein the sulfonation process comprises sonication, optionally with stirring, while the PAEK material is exposed to the sulfuric acid;
optionally, following sulfonation and prior to exposing the PAEK material to water, subjecting the PAEK material to a physical cleaning process to remove at least a portion of excess sulfuric acid from the PAEK material; and
subjecting the PAEK material to a hydrothermal process comprising contacting the PAEK material with water at a temperature of 40° C. to 80° C. for a time of 1 to 5 hours to cause substantial de-sulfonation of the PAEK material.

16. The method of claim 15, the method comprising subjecting the PAEK material to a physical cleaning process to remove at least a portion of excess sulfuric acid from the PAEK material, wherein the physical cleaning process comprises using compressed air to remove at least a portion of the excess sulfuric acid from the PAEK material.

17. The method of claim 15, wherein the PAEK material comprises one or more of polyetheretherketone (PEEK), polyetherketone (PEK), polyetherketoneketone (PEKK), polyetheretherketoneketone (PEEKK), or polyetherketoneetherketoneketone (PEKEKK).

18. The method of claim 15, wherein a treated surface of the PAEK material exhibits (i) a pore coverage (% area) of 5% or greater as a result of the method and (ii) an average pore diameter of 80 μm or greater as a result of the method.

19. An implantable device comprising a PAEK material manufactured using the method of claim 15.

20. The implantable device of claim 19, wherein the PAEK material is a spinal fusion cage.
